(19) United States
(12) Patent Application Publication
(10) Pub. No.: US 2026/0068905 A1
(43) Pub. Date: Mar. 12, 2026

(54) SWEET COCOA POWDERS

(71) Applicant: Cargill, Incorporated, Wayzata, MN (US)

(72) Inventors: Vincent Schoot Uiterkamp, Haarlem (NL); Stijn Folkert Boonstra, Utrecht (NL)

(73) Assignee: Cargill, Incorporated, Wayzata, MN (US)

(21) Appl. No.: 19/105,848

(22) PCT Filed: Aug. 28, 2023
(86) PCT No.: PCT/US2023/072973

§ 371 Date: Feb. 24, 2025

(30) Foreign Application Priority Data
Aug. 30, 2022 (EP) 22193023.3

(51) Int. Cl. A23G 1/30; A23G 1/00; A23G 1/02; A23L 2/52; A23L 33/105; A61K 8/92; A61Q 19/00

(52) U.S. Cl. CPC A23G 1/30; A23G 1/002; A23G 1/0023; A23G 1/02; A23L 2/52; A23L 33/105; A61K 8/922; A61Q 19/00; A61K 2800/805

(57) ABSTRACT

The present disclosure relates to an extracted cocoa material that has less bitter flavor and a sweeter flavor compared to an equivalent non-extracted cocoa material. The present disclosure also relates to a use of the extracted cocoa material to prepare a sugar-reduced or sugar-free food or beverage product. A method of preparing an extracted cocoa material is also described herein.

### CROSS-REFERENCE TO RELATED APPLICATIONS

This application claims the benefit of European Application No. 22193023.3, filed Aug. 30, 2022, which is incorporated by reference herein in its entirety.

### FILED OF THE INVENTION

The invention relates to cocoa materials, to uses of cocoa materials, to method for preparing the cocoa materials, and to food or beverage products comprising the cocoa materials.

### BACKGROUND

Cocoa beans are processed in large volumes into a wide variety of chocolate-related products. Typically, cocoa beans are initially fermented and roasted before being processed into cocoa nibs and then into cocoa liquor, which then is further processed to separate the liquor into its cocoa butter and cocoa powder constituents. To separate cocoa butter from cocoa powder, conventional mechanical pressing of the cocoa liquor is typically used. The result of mechanical pressing is a press cake of cocoa solids which is further milled to produce cocoa powder.

Cocoa butter, cocoa liquor and/or cocoa powder can be used in many products, including chocolate products (such as chocolate bars, coatings, fillings, and inclusions), bakery products (such as cakes, cookies, and pre-mixes), and beverages (such as drinking chocolate, and chocolate-flavored milk).

However, cocoa nibs are rich in alkaloids such as theobromine, trigonelline, nicotinic acid, paraxanthine, and caffeine which give products derived from cocoa nibs (such as cocoa powder) a sharp, bitter flavor. This bitterness is exacerbated by traditional processing techniques such as roasting in which alkylpyrazines form. As a result, products that are flavored or colored with cocoa powder typically have to have a high sugar content to mask the bitter flavor of the cocoa.

In addition to providing sweetness, sugar also provides bulk and plays a significant role in the structure, volume, and mouthfeel of the finished product. However, there is a growing pressure on the food industry to use less sugar in order to reduce sugar consumption and associated health issues in the global population. Several countries have enacted laws requiring the sugar content of commercial foods to be below a certain threshold. Formulating foods without sugar or with reduced sugar content is challenging because a sugar replacement will ideally not only replace the sweetness of sugar, but also fulfil all its other various functions.

United States Publication Number US2022/0232849 describes a reduced sugar milk chocolate wherein at least a portion of the unsweetened chocolate used to make the product has been subjected to a debittering process. The debittering process is a water extraction process and results in a flavorless cocoa powder. The water extraction process removes a majority of theobromine, caffeine, sugar and chocolate flavor.

JP H10 10179078A relates to extracting flavor components from cocoa raw material and describes an extraction process with addition of water or ethanol and extracting with liquefied carbon dioxide. The water extracted flavor components are used as food additives and for flavor enhancement.

United States Publication Number 2001/0007690A1 relates to preparing hypoallergenic and reduced fat foods. The process uses super critical carbon dioxide resulting in a defatted cocoa powder and the process causes a change in the structure of protein making the resulting product hypoallergenic. The process is conducted between about 40° C. and about 60° C.

KR 101836348B1 relates to an cocoa extract for cosmetics. The process includes adding liquid carbon dioxide and adding alcohol after the extraction of the fat component. The process includes an extraction temperature of 38 to 42° C.

Replacing sugar with additional cocoa solids has typically not been possible as it would result in an unpalatably bitter product. There therefore remains a need in the industry to replace sugar content in cocoa-containing products without the undesirable side effect of imparting bitterness.

### SUMMARY

The present disclosure provides an extracted cocoa material, preferably an extracted cocoa powder, that has less bitter flavor, and preferably a sweeter flavor, compared to an equivalent non-extracted cocoa material.

The present disclosure also provides a food or beverage product that comprises the extracted cocoa material of the present invention.

The present disclosure also provides a use of the extracted cocoa material to prepare sugar-reduced or sugar-free food or beverage products.

The present disclosure also provides a method for reducing bitter flavor and/or increasing sweet flavor in a cocoa material. The method comprises the steps of providing a cocoa material, preferably a cocoa nib; extracting the cocoa material with a liquefied gas, preferably liquefied carbon dioxide, at an extraction temperature and an extraction pressure to obtain an extracted cocoa material and, optionally, an extraction butter; and processing the extracted cocoa material to yield a reduced bitterness extracted cocoa powder.

The present disclosure also provides an extraction butter prepared by the method as described herein. The extraction butter is rich in antioxidants and preferably has not been subjected to heat or alkali treatment. A personal care product comprising the extraction butter is also provided.

Representative features of the present invention are set out in the following clauses, which stand alone or may be combined, in any combination, with one or more features disclosed in the text of the Specification.

The present invention is as set out in the following clauses:

Clause 1: An extracted cocoa material having less bitter flavor, and preferably a sweeter flavor, compared to an equivalent non-extracted cocoa material.

Clause 2: The extracted cocoa material of clause 1, wherein the content of one or more bitter components in the extracted cocoa material is lower compared to an equivalent non-extracted cocoa material.

Clause 3: The extracted cocoa material of any of the preceding clauses, wherein the one or more bitter components are selected from a group consisting of alkaloid, theobromine, caffeine, polyphenol, flavanol, epicatechin, catechin, epigallocatechin, proanthocianidin, and any combinations thereof.

Clause 4: The extracted cocoa material of any of the preceding clauses, which has a lower alkaloid content, preferably a lower theobromine and/or caffeine content, compared to an equivalent non-extracted cocoa material.

Clause 5: The extracted cocoa material of any of the preceding clauses, which has a lower polyphenol content compared to an equivalent non-extracted cocoa material.

Clause 6: The extracted cocoa material of any of the preceding clauses, which has a lower flavanol content compared to an equivalent non-extracted cocoa material.

Clause 7: The extracted cocoa material of any of the preceding clauses, which is an extracted cocoa powder.

Clause 8: The extracted cocoa material of any of the preceding clauses, which is an extracted cocoa nib, an extracted cocoa liquor, or an extracted cocoa cake and which has a fat content of 10-35 wt %, preferably 15-30 wt %, based on a total dry weight of the extracted cocoa material.

Clause 9: A food or beverage product comprising the extracted cocoa material of any one of clauses 1 to 8, and preferably the extracted cocoa powder of claim 7.

Clause 10: Use of the extracted cocoa material of any of the clauses 1 to 8, preferably of the extracted cocoa powder of clause 7, to prepare sugar-reduced or sugar-free food or beverage products.

Clause 11: A method for reducing bitter flavor and/or increasing sweet flavor in a cocoa material, comprising the steps of:
a. providing a cocoa material, preferably a cocoa nib;
b. extracting the cocoa material with a liquefied gas, preferably liquified carbon dioxide, at an extraction temperature and an extraction pressure to obtain an extracted cocoa material and, optionally, an extraction butter; and
c. processing the extracted cocoa material to yield an extracted cocoa powder;
wherein the extracted cocoa powder has a less bitter flavor, and preferably a sweeter flavor, compared to an equivalent non-extracted cocoa powder.

Clause 12: The method of clause 11, wherein the liquefied gas, preferably liquified carbon dioxide, and the cocoa material are used in a ratio of between 30:1 to 100:1.

Clause 13: The method of any one of clauses 11 to 12, wherein the extraction temperature is from 60° C. to 120° C.

Clause 14: The method of any one of clauses 11 to 13, wherein the extraction pressure is from 100 bar to 800 bar.

Clause 15: The extraction butter obtainable by the method of any one of clauses 11 to 14, which is rich in antioxidants and preferably has not been subjected to heat or alkali treatment.

Clause 16: A personal care product comprising the extraction butter of clauses 15.

### BRIEF DESCRIPTION OF THE FIGURES

The drawings illustrate generally, by way of example, but not by way of limitation, various aspects discussed in the present document.

FIGS. FIGS. 1A 1Fshow observations of a sensory testing on attributes of sampled cocoa powders.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Unless defined otherwise, all technical and scientific terms used herein have the same meaning as commonly understood by one skilled in the art to which this invention belongs. As used herein, each of the following terms has the meaning associated with it as defined below.

Unless otherwise indicated, the word “a” in the present application is not exclusively limited to mean “only one” but inclusively to mean “at least one.”

Unless otherwise indicated, the term “cocoa nibs” is used to refer to pieces of cocoa bean from which substantially all the shell has been removed. The nibs may come from beans with any degree of fermentation (including under-fermented or unfermented beans). They will typically be sterilized, and they may be roasted, alkalized and/or treated in any other way.

Unless otherwise indicated, the term “cocoa mass” is used to refer to a material which includes cocoa fat and cocoa solids and is typically obtained by grinding cocoa nibs to form a homogenous mixture. The term “cocoa liquor” is synonymous with the term “cocoa mass” and will be used interchangeably therewith.

Unless otherwise indicated, the phrase “suitable for human consumption” means that the composition is generally recognized as safe for eating and drinking and as not being toxic, poisonous, or otherwise harmful to humans when used in functionally useful amounts in solid and liquid food products.

Unless otherwise indicated, the phrases “uniform mixing” or “uniformly mixed” means that the majority of particles are coated with at least some dispersing agent.

Background references disclosing cocoa technology include, for example, Industrial Chocolate Manufacture and Use. 2nd Ed., Ed. S. T. Beckett, Blackie Academic, 1994; and Chocolate, Cocoa, and Confectionery: Science and Technology, 3rd Ed., B. Minifie, Nan Nostrand, 1989. For example, these references discuss methods for converting raw cocoa products into cocoa mass and cocoa liquor.

Unless expressly stated, ppm (parts per million), percentage, and ratios are on a by weight basis. Percentage on a by weight basis is also referred to as wt % below.

The term “for example,” “for instance,” “such as,” or “including” as used herein is meant to introduce examples that further clarify more general subject matter. Unless otherwise specified, these examples are provided only as an aid for understanding the applications illustrated in the present disclosure and are not meant to be limiting in any fashion.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, “room temperature” or “RT” refers to a temperature between 20° C. to 25° C.

The present application is directed to novel cocoa products produced by a mild liquefied gas extraction of cocoa materials such as cocoa nibs.

Liquefied gas extraction is known as an alternative to mechanical pressing to separate cocoa liquor into its cocoa butter and cocoa powder constituents as disclosed in, for example, U.S. Pat. Nos. 6,066,350 and 6,361,814. In a preferred example of this process, cocoa liquor is mixed with liquefied, pressurized butane to form a slurry, and the slurry is subjected to separation on, for example, a belt filter. The belt filter retains wet cocoa solids as solvent passes through the belt filter extracting cocoa butter. The remaining wet cocoa solids, which usually retain some cocoa butter, are processed into cocoa powder by, among other things, breaking up any clumped particles and removing solvent (desolventization).

### Extraction Process

The process of the present application is directed to a novel method for preparing cocoa products comprising the step of extracting cocoa materials, preferably cocoa nibs, with a liquefied gas as solvent, preferably liquified carbon dioxide, at an extraction temperature and extraction pressure. After extraction with the liquified gas, two cocoa products can be recovered: the extracted cocoa material and the extraction butter (recovered cocoa butter extracted from the cocoa material). Depending on the nature of the starting material, the extracted cocoa material may include extracted cocoa nibs, extracted cocoa liquor, extracted cocoa cake, and extracted cocoa powder. Preferably, the starting material will be selected from cocoa beans and/or cocoa nibs, and the extracted cocoa material can be further processed (e.g., by milling, pressing, and/or grinding) to produce extracted cocoa nibs, extracted cocoa liquor, extracted cocoa cake, and/or extracted cocoa powder. The extracted cocoa materials, and in particular extracted cocoa powders, were surprisingly found to have a less bitter flavor, and preferably a sweeter flavor, compared to equivalent non-extracted cocoa materials. Advantages include less bitter flavor, less sugar needed to mask bitterness in the extracted cocoa materials, and the possibility of using the extracted cocoa powder as a bulking agent to replace sugar content in finished products.

The liquefied solvent used in the liquefied gas extraction can be, for example, saturated hydrocarbons including propane, butane, pentane, and all isomers thereof including n-butane, isobutane, n-propane, n-pentane, and isopentane, and preferably liquified carbon dioxide. In one aspect, the ratio of liquefied gas, preferably liquefied carbon dioxide, to cocoa material used in the process of the present invention is between 30:1 and 100:1, preferably between 40:1 and 90:1, more preferably between 50:1 and 80:1, more preferably between 60:1 and 80:1, more preferably between 70:1 and 80:1, more preferably between 60:1 and 85:1, or more preferably between 70:1 and 85:1.

The extraction temperature can be, for example, 60-120° C., preferably 65-110° C., more preferably 70-100° C., more preferably 75-90° C., more preferably 60-80° C., or more preferably 80-120° C. The extraction pressure can be, for example, 100-800 bar, preferably 200-700 bar, more preferably 300-600 bar, more preferably 400-500 bar, more preferably 100-400 bar, or more preferably 400-800 bar.

In one aspect, a method of reducing bitter flavor and/or increasing sweet flavor in a cocoa powder is also provided in the present invention. The method comprises steps of providing a cocoa material, preferably a cocoa nib; extracting the cocoa material with a liquefied gas, preferably liquified carbon dioxide, at an extraction temperature and an extraction pressure to obtain an extracted cocoa material and, optionally, an extraction butter; and processing the extracted cocoa material to yield an extracted cocoa powder. The extracted cocoa material has a less bitter flavor, and preferably a sweeter flavor, compared to an equivalent non-extracted cocoa material.

### Cocoa Material

Cocoa beans, cocoa nibs, cocoa mass, cocoa cake, and cocoa powder are examples of cocoa material that can be used as the starting material for the extraction process of the present invention. Preferably, the starting material will be cocoa nibs.

After extraction, the extracted cocoa material can be further processed and desolventized to yield an extracted cocoa powder. When liquefied carbon dioxide is used as solvent, a separate desolventization step may not be needed as any residual liquefied carbon dioxide present in the extracted cocoa material would naturally evaporate out. When a liquefied gas other than liquefied carbon dioxide is used as solvent, a separate desolventization step may be needed to remove any residual solvent present in the extracted cocoa material.

### Extracted Cocoa Material

The extracted cocoa material may optionally be ground, milled and/or pressed to produce an extracted cocoa powder of the invention. For example, if the starting cocoa material includes cocoa nibs, these will be ground to a liquor, then pressed to extract cocoa butter. The resulting cocoa cake will then be further milled to produce the extracted cocoa powder. In one aspect, the molecular size of over 99.5% of the extracted cocoa powder particles will be below 75 microns.

The extracted cocoa powders of the present invention can be made from cocoa beans or cocoa nibs which have not been alkalized. In this case, the extracted cocoa powders are referred to as natural extracted cocoa powder. However, the extracted cocoa powders can also be made from an alkalized cocoa material (e.g., alkalized cocoa beans, alkalized nibs, or alkalized cocoa mass), resulting in alkalized or “dutched” extracted cocoa powders.

The extracted cocoa powders can be checked for quality including checked for color, flavor, fat percentage, moisture percentage, pH, ash on fat-free dry, alkalinity of the ash, shell percentage based on nib, percentage sieve residue on 75 microns, traces of heavy metals, pesticide residues, and microbiology.

Advantageously, the extracted cocoa powders of the present invention will have a sweeter flavor compared to an equivalent non-extracted cocoa powder. The term “equivalent non-extracted cocoa powder” as used herein means a powder produced from the same starting material (e.g., from cocoa beans of the same origin and same batch) which have been treated in the same way (e.g., same degree of fermentation, if any, same roasting time and conditions, if any, and same kind and degree of alkalization, if any) except that it has not been subjected to the extraction process of the present invention.

Advantageously, the extracted cocoa powders of the present invention will have a less bitter flavor compared to an equivalent non-extracted cocoa powder. In other words, the content of one or more bitter components in the extracted cocoa powders of the present invention is lower compared to an equivalent non-extracted cocoa powder. Bitter components are substances present in cocoa beans that impart a bitter flavor or bitterness to the cocoa beans. Examples of one or more bitter components include alkaloids, for example, theobromine, caffeine, and their derivatives, polyphenols, flavanols, for example, epicatechin, catechin, epigallocatechin, proanthocianidin, and their derivatives, or any combinations thereof.

In one aspect, the extracted cocoa powders of the present invention will have a lower alkaloid content, preferably a lower theobromine and/or caffeine content, compared to an equivalent non-extracted cocoa powder.

Advantageously, the extracted cocoa powders of the present invention will have an alkaloid level that is at least 10%, preferably at least 20%, more preferably at least 30%, more preferably at least 40%, more preferably at least 50%, more preferably at least 60%, more preferably at least 70%, or more preferably at least 80% lower than the alkaloid content of an equivalent non-extracted powder. They will preferably have a theobromine content that is at least 10%, preferably at least 20%, more preferably at least 30%, more preferably at least 40%, more preferably at least 50% lower, more preferably at least 60%, more preferably at least 70%, or more preferably at least 80% than the theobromine content of an equivalent non-extracted powder. For example, the theobromine content may be reduced by 60% or more, preferably by 70% or more, or more preferably by 80% or more. In addition, or alternatively, they will preferably have a caffeine content that is at least 10%, preferably at least 20%, more preferably at least 30%, more preferably at least 40%, more preferably at least 50%, more preferably at least 60%, more preferably at least 70%, or more preferably at least 80% lower than the caffeine content of an equivalent non-extracted powder. For example, the caffeine content may be reduced by 60% or more, preferably by 70% or more, or more preferably by 80% or more.

In one aspect, the extracted cocoa powders of the present invention will have a lower polyphenol content compared to an equivalent non-extracted cocoa powder.

Advantageously, the extracted cocoa powders of the present invention will have a polyphenol level that is at least 10%, preferably at least 20%, more preferably at least 30%, more preferably at least 40%, more preferably at least 50%, more preferably at least 60%, more preferably at least 70%, or more preferably at least 80% lower than the polyphenol content of an equivalent non-extracted powder.

In one aspect, the extracted cocoa powders of the present invention will have a lower flavanol content compared to an equivalent non-extracted cocoa powder. The flavanol can be selected from a group consisting of epicatechin, catechin, epigallocatechin, and proanthocianidin.

Advantageously, the extracted cocoa powders of the present invention will have a flavanol level that is at least 10%, preferably at least 20%, more preferably at least 30%, more preferably at least 40%, more preferably at least 50%, more preferably at least 60%, more preferably at least 70%, or more preferably at least 80% lower than the flavanol content of an equivalent non-extracted powder. They will preferably have an epicatechin content that is at least 10%, preferably at least 20%, more preferably at least 30%, more preferably at least 40%, more preferably at least 50%, more preferably at least 60%, more preferably at least 70%, or more preferably at least 80% lower than the epicatechin content of an equivalent non-extracted powder. In addition, or alternatively, they will preferably have a catechin content that is at least 10%, preferably at least 20%, more preferably at least 30%, more preferably at least 40%, more preferably at least 50% lower, more preferably at least 60%, more preferably at least 70%, or more preferably at least 80% than the catechin content of an equivalent non-extracted powder. In addition, or alternatively, they will preferably have an epigallocatechin content that is at least 10%, preferably at least 20%, more preferably at least 30%, more preferably at least 40%, more preferably at least 50%, more preferably at least 60%, more preferably at least 70%, or more preferably at least 80% lower than the epigallocatechin content of an equivalent non-extracted powder. In addition, or alternatively, they will preferably have a proanthocianidin content that is at least 10%, preferably at least 20%, more preferably at least 30%, more preferably at least 40%, more preferably at least 50%, more preferably at least 60%, more preferably at least 70%, or more preferably at least 80% lower than the proanthocianidin content of an equivalent non-extracted powder.

The fat content of the extracted cocoa material will typically have been reduced, relative to the starting cocoa material, by no more than 35 wt %, preferably by no more than 30 wt %, more preferably by no more than 25 wt %, more preferably by no more than 20 wt %, more preferably by no more than 15 wt %, or more preferably by no more than 10 wt %, for example by 10-35 wt %, preferably by 15-30 wt %, or more preferably by 20-25 wt %, based on the total dry weight of the extracted cocoa material. In other words, the extracted cocoa material will have a fat content of at least 10 wt %, preferably at least 15 wt %, more preferably at least 20 wt %, more preferably at least 25 wt %, more preferably at least 30 wt %, or more preferably at least 35 wt %, for example, approximately 10-35 wt %, or preferably 15-30 wt %, based on the total dry weight of the extracted cocoa material.

As used herein, “taste” refers to sensory perception on the tongue. For example, the five basic tastes are sweet, sour, salty, bitter, and umami.

As used herein, “aroma” refers to the orthonasal perception in the nasal cavity.

As used herein, “flavor” refers to the taste and retronasal perception in the nasal cavity.

As used herein, “off-taste(s)” refer to a taste or flavor attribute profile that is not characteristic or usually associated with a substance or composition as described herein and/or a characteristic taste or flavor associated with a substance or composition that is undesirable. For example, the off-taste may be an undesirable taste such as bitterness, undesirable mouthfeel such as astringency, mouth drying, undesirable flavor such as rancid, cardboard, aftertaste, inconsistent flavor (e.g., a flavor with an uneven onset or intensity, a flavor that may be perceived too early or too late), and the like.

A sensory panel can be used to determine the magnitude of, for example, reduction in bitterness or shifts in its temporal profile. Sensory panels are a scientific and reproducible method that is essential to the food and beverage industry. A sensory panel involves a group of two or more individual panelists. Panelists are instructed according to industry-recognized practices to avoid the influence of personal subjectivity and strengthen reproducibility. For example, panelists may objectively evaluate sensory attributes of a tested product but may not provide subjective attributes such as personal preference. In various aspects, the sensory panel can be conducted with two, three, four, five, six, or more panelists, in which the panelists identify and agree on a lexicon of sensory attributes for a given set of samples. After evaluating a specific sample, the panelists can assign a numerical intensity score for each attribute using an intensity scale. For example, intensity scales can range from 0 to 6 (i.e., 0=not detected, 1=trace, 2=slight, 3=moderate, 4=definite, 5=strong, 6=extreme), 0 to 9 (i.e., 0=not detected, 1=trace, 2=faint, 3=slight, 4=mild, 5=moderate, 6=definite, 7=strong, 8=very strong, 9=extreme), or 0 to 15, where 0 corresponds to the absence of the attribute, while 6, 9, or 15, respectively, corresponds to the upper bound extreme occurrence of the attribute. The panel may use a roundtable consensus approach, or the panelists may score and evaluate the sensory attribute(s) individually. Either format can further involve a panel leader who directs the discussion regarding terminology and directs the panel to evaluate particular products and attributes. In other aspects, a trained sensory panel can be utilized to assess specific attributes using descriptive analysis or time intensity methodologies.

As used herein, a “panelist” refers to a highly trained expert taster, such as one commonly used for sensory methodologies (e.g., descriptive analysis), and/or an experienced taster familiar with the sensory attribute(s) being tested. In some aspects, the panelist may be a trained panelist. A trained panelist has undergone training to understand the terms and sensory phenomenon associated with those sensory attributes relevant to the tested product and are aligned on the use of common descriptors for those sensory attributes of interest (i.e., a sensory lexicon). For example, a trained panelist testing a given composition will understand the terms and sensory attributes associated with said composition, e.g., saltiness, sourness, bitterness, astringency, mouthfeel, acidity, and the like. The trained panelist will have been trained against reference samples corresponding to the sensory attributes being tested and thus have calibrated to recognize and quantitatively assess such criteria. In some aspects, the panelist may be an experienced taster.

As used herein, “roundtable consensus approach” refers to the sensory panel assay methodology wherein panelists discuss sensory attributes and intensities before mutually agreeing on an intensity score and attribute characterization for the particular sensory attribute(s) being assayed. A sensory panel using a roundtable consensus approach may include 2, 3, 4, 5, 6, or more panelists. Consensus intensity scales can range from 0 to 6 (i.e., 0=not detected, 1=trace, 2=slight, 3=moderate, 4=definite, 5=strong, 6=extreme) or 0 to 9 (i.e., 0=not detected, 1=trace, 2=faint, 3=slight, 4=mild, 5=moderate, 6=definite, 7=strong, 8=very strong, 9=extreme). For a given set of samples, the panelists will identify and agree on a lexicon of sensory attribute, including, if applicable, reference or standardized samples (also referred to as sensory anchors) for a particular sensory attribute. The reference sample(s) used for a given sensory attribute(s) will depend on the samples being assayed and the lexicon of sensory attributes determined by the panel. One of skill in the art will recognize the appropriate lexicon and reference or standard samples necessary for sensory assessment of a given sample(s).

In some aspects, the samples are scored and evaluated by panelists independently after panelists have agreed upon or been instructed in a lexicon of sensory attributes and intensity scores including, if applicable, assay specific calibration on reference samples (also referred to as sensory anchors) for a particular sensory attribute. Examples of common reference samples are described below. Panelists may evaluate samples in replicate and may be blinded to the samples they are testing. Samples being tested may be provided to the panelists randomly or in a sequential order. In some aspects, samples may be tested by panelists using a randomized balanced sequential order. Scores from individual panelists are then assessed using standard statistical analysis methods to determine an average sensory intensity score. One of skill in the art will recognize the appropriate lexicon and reference or standard samples necessary for sensory assessment of a given sample(s) as well as the appropriate statistical analysis methods.

As used herein, “randomized balanced sequential order” refers to the order in which samples are presented in which the order is randomized but across all panelists all possible orders of the samples will be presented to remove bias for the samples being tested in a particular order. For example, for a randomized balanced sequential order of two samples, there would be an equal likelihood that a given panelist receives sample 1 before sample 2 and sample 2 before sample 1. In an example with three samples (i.e., samples 1, 2, and 3), a randomized balanced sequential order would include an equal likelihood that panelists receiving samples in the following orders: (i) 1, 2, 3; (ii) 1, 3, 2; (iii) 2, 1, 3; (iv) 2, 3, 1; (v) 3, 2, 1; (vi) 3, 1, 2.

A sensory attribute(s) of a given composition may be evaluated in comparison to one or more reference or anchor samples. For example, sucrose solutions can be used by experienced panelists as sweetness anchors to assess the relative intensity of sweetness for a given composition; and caffeine solutions can be used by experienced panelists as bitterness anchors to assess the relative intensity of bitterness for a given composition. Experienced panelists can be presented with a solution to assess sensory attributes, e.g., 10-20 mL of a sample. Panelists will dispense approximately 3-4 mL of each solution into their own mouths, disperse the solution by moving their tongues, and record a value for the particular sensory attribute being tested. If multiple solutions are to be tested in a session, the panelists may cleanse their palates with water between samples. For example, a roundtable assessment of saltiness, sweetness, sourness, umami, and the like can assign a scale of 0 to 9 with, e.g., a score of 0 indicating no saltiness and a score of 9 indicating extreme saltiness (0=not detected, 1=trace, 2=faint, 3=slight, 4=mild, 5=moderate, 6=definite, 7=strong, 8=very strong, 9=extreme). Equivalent scales and methodologies can be used for sweet and bitter sensory attributes.

Bitterness of a composition can be tested by a panel of at least two panelists. The panelists can use a standard range of 0.0125% (wt), 0.01875% (wt), 0.025% (wt), 0.031% (wt), 0.07% (wt), and 0.12% (wt) caffeine solutions in water corresponding to a bitterness intensity value of 2, 3, 4, 5, 10, and 15, respectively. A skilled artisan will recognize that depending on the sample/composition being tested, the number and range of standard solutions may be changed (e.g., using only the solutions corresponding to the 2, 3, and 5 bitterness intensity values). For each test composition, the panelists dispense approximately 2-5 mL, for liquid compositions or solutions prepared with water, or 5-10 g, for solid compositions, of each composition into their own mouths, disperses the composition by moving their tongues/chewing, and records a bitterness intensity value between 0 and 15 for each composition based on comparison to the aforementioned standard caffeine solutions. Between tasting compositions, the panelists are able to cleanse their palates with water. The panelists also can taste the standard 0.0125%, 0.01875%, 0.025%, 0.031%, 0.07%, and 0.12% caffeine solutions ad libitum between tasting test solutions to ensure recorded bitterness intensity values are accurate against the scale of the standard caffeine solutions. The temperature at which the test is conducted may be specific to the sample beginning tested, e.g., samples may be tested at 22° C. (e.g., room temperature), at 0° C. (e.g., for frozen samples), or between 60-80° C. (e.g., a cooked sample served warm). One skilled in the art will recognize the appropriate temperature for testing a given sample. This test is referred to herein as the “Standardized Bitterness Intensity Test.”

Sweetness of a composition can be tested by a panel of at least two panelists. The panelists can use a standard range of 2% (wt), 5% (wt), 8% (wt), 10% (wt), and 15% (wt) sucrose solutions corresponding to a sweetness intensity value of 2, 5, 8, 10, and 15, respectively. A skilled artisan will recognize that depending on the sample/composition being tested, the number and range of standard solutions may be changed (e.g., using only the solutions corresponding to the 2, 5, and 8 sweetness intensity values). For each test composition, the panelists dispense approximately 2-5 mL, for liquid compositions or solutions prepared with water, or 5-10 g, for solid compositions, of each composition into their own mouths, disperses the composition by moving their tongues/chewing, and records a sweetness intensity value between 0 and 15 for each composition based on comparison to the aforementioned standard sucrose solutions. Between tasting compositions, the panelists are able to cleanse their palates with water. The panelists also can taste the standard 2%, 5%, 8%, 10%, and 15% sucrose solutions ad libitum between tasting test solutions to ensure recorded sweetness intensity values are accurate against the scale of the standard sucrose solutions. The temperature at which the test is conducted may be specific to the sample beginning tested, e.g., samples may be tested at 22° C. (e.g., room temperature), at 0° C. (e.g., for frozen samples), or between 60-80° C. (e.g., a cooked sample served warm). One skilled in the art will recognize the appropriate temperature for testing a given sample. This test is referred to herein as the “Standardized Sweetness Intensity Test.”

This disclosure is not limited to sensory testing by experienced or trained panelists. For example, it is possible to utilize untrained and inexperienced panelists. However, in the case of untrained and inexperienced panelists, a greater number of these panelists is usually necessary to provide reproducible results, which will typically focus on subjective attributes such as preference or overall liking. Similarly, untrained, and inexperienced panelists may be asked to evaluate relative changes in a given sensory attribute between two samples. For example, if a particular sample is more or less sweet, more or less bitter, etc., than a reference sample.

Conventional complementary ingredients can be included with the extracted cocoa powder if desired. For example, the extracted cocoa powder also can be mixed with sweetening agents such as, for example, natural sweeteners such as, for example, sugars such as sucrose, sugar alcohols such as xylitol, sorbitol, erythritol, artificial sweeteners such as, for example, saccharin and aspartame, sodium cyclamate, and mixtures of sodium cyclamate and saccharin. Other examples include acesulfame-k, alitame, and sucralose. Mixtures can be used. Milk powders also can be used. Other examples of complementary ingredients include vitamins, trehalose, colors, flavors, and bulking agents. Complementary ingredients can be selected to be compatible with the solvent.

### Applications

The extracted cocoa materials of the present invention can advantageously be used in food and beverage products, in particular in sugar-reduced or sugar-free food and beverage products, without causing undesirable bitterness. Thus, the present invention also provides food or beverage products comprising the extracted cocoa materials, and/or preferably the extracted cocoa powders as described herein.

The extracted cocoa materials may be used alone, or in addition to/in admixture with other non-extracted cocoa materials. The quantities to be used will depend on the desired end product and will readily be determined by a skilled person. Advantageously, the extracted cocoa material can be used to replace some or all of a product's (non-extracted) cocoa material content or in addition thereto. For example, extracted cocoa liquor could be used to replace some or all of the (standard) non-extracted cocoa liquor content in chocolate. In another example, extracted cocoa powder could be used as a substitute for some or all of the non-extracted cocoa content or in addition to it. Preferably, the extracted cocoa material will be used to replace equal parts of non-extracted cocoa material or in addition to non-extracted cocoa materials.

It has surprisingly been found that the extracted cocoa materials of the present invention not only impart a significantly less bitter flavor, meaning they can be used in foods and beverages without increasing bitterness, they also impart a desirable sweeter flavor. This means that they can be used to replace some or all of the sugar content in food or beverage compositions. Advantageously, they also provide bulk and volume which is important when removing sugar content.

### Extraction Butter

As described above, the process of the present invention results in extracted cocoa materials with a reduced fat content. Thus, another product of the extraction process is the extraction butter which can be recovered separately from the extracted cocoa materials. Advantageously, the extraction butter will be rich in antioxidants (such as theobromine) and will have a distinct chocolatey flavor not normally associated with cocoa butter. Advantageously, since the process of the invention may be carried out on non-alkalized and non-roasted nibs, the extraction butter will not have been damaged by either heat treatment or alkalization.

Thus, in another aspect of the present invention, there is provided a cocoa butter which is rich in antioxidants and which preferably has not been subjected to heat or alkali treatment.

The cocoa butter of the present invention can be used in food and beverage products but can advantageously also be used in personal care products, cosmetic products, skin care products, sun care products, hair care products (e.g., shampoo, conditioner), make-up products. Thus, the present invention also provides personal care products comprising the extracted cocoa butter as described herein.

As used herein, “personal care product” means and comprises any cosmetic, hygienic, toiletry, skin care, and topical care products including, without limitation, leave-on products (i.e., products that are left on keratinous substrates after application); rinse-off products (i.e., products that are washed or rinsed from keratinous substrates during or within a few minutes of application); shampoos; hair curling and hair straightening products; hair style maintaining and hair conditioning products; lotions and creams for nails, hands, feet, face, scalp and/or body; hair dye; face and body makeup; nail care products; astringents; deodorants; antiperspirants; anti-acne; antiaging; depilatories; colognes and perfumes; skin protective creams and lotions (such as sunscreens); skin and body cleansers; skin conditioners; skin toners; skin firming compositions; skin tanning and lightening compositions; liquid soaps; bar soaps; bath products; and shaving products.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### Example 1—Study on Different Process Conditions

### Materials and Method

Tests were carried out to study different process conditions, as shown in Table 1, for extracting cocoa nibs by liquified carbon dioxide. In all the tests, about 78 kg liquified carbon dioxide per kilogram of cocoa nibs was used.

TABLE 1

| | Pressure | Temperature | Time |
|---|---|---|---|
| Test No. | (bar) | (° C.) | (hour) |
| 1 | 800 | 80 | 7 |
| 2 | 800 | 80 | 16 |
| 3 | 800 | 80 | 11 |
| 4 | 500 | 80 | 11 |
| 5 | 500 | 80 | 7 |

First, deshelled cocoa nibs were loaded into an extraction vessel. Liquified carbon dioxide at pressures and temperatures in Table 1 was pumped through the cocoa nibs inside the extraction vessel. Extracted cocoa material, extraction butter, and bitter components were extracted from the cocoa nibs and collected.

### Results

Results of the tests are shown in Table 2. Fat content in the cocoa nibs was presumed to be 45 wt % based on the total dry weight of the cocoa nibs. The fat content in the extracted cocoa material was calculated based on other mass streams (e.g., the amount of fat that was not in the extracted cocoa material, as percentage of the solid fractions).

TABLE 2

| | | | | Fat content | | | | | |
|---|---|---|---|---|---|---|---|---|---|
| | Mass (kg) | | | | | Extracted | Extracted | | |
| | | Extracted | | Cocoa | Cocoa | cocoa | cocoa | Extraction | Extraction |
| Test | Cocoa | cocoa | Extraction | nibs | nibs | material | material | butter | butter |
| No. | nibs | material | butter | (wt %) | (kg) | (wt %) | (kg) | (wt %) | (kg) |
| 1 | 21 | 13.5 | 7.5 | 45 | 9.45 | 14.44 | 1.95 | 100 | 7.50 |
| 2 | 21 | 14.6 | 6.52 | 45 | 9.45 | 20.07 | 2.93 | 100 | 6.52 |
| 3 | 21 | 13.9 | 7.38 | 45 | 9.45 | 14.89 | 2.07 | 100 | 7.38 |
| 4 | 21 | 14 | 5.31 | 45 | 9.45 | 29.57 | 4.14 | 100 | 5.31 |
| 5 | 21 | 13.5 | 5.82 | 45 | 9.45 | 26.89 | 3.63 | 100 | 5.82 |

### Example 2—Sensory Testing

An informal sensory panel with eight untrained panelists sampled eight different cocoa powders and rated the samples for attributes of cocoa flavor, sweetness, bitterness, degree of roast, degree of burnt, off-flavors, and astringency. Table 3 shows profiles of the six sampled cocoa powders, in which Samples #3, #5, and #6 include extracted cocoa powders of the present invention.

TABLE 3

| Cocoa Powder | |
|---|---|
| Sample | Description |
| 1 | Fat-reduced alkalized cocoa powders |
| 2 | Blend of natural cocoa powders (40 wt %) and highly |
| | alkalized cocoa powders (60 wt %) |
| 3 | Blend of extracted cocoa powders (40 wt %) and highly |
| | alkalized cocoa powders (60 wt %) |
| 4 | Blend of natural cocoa powders (30 wt %) and standard |
| | mediumly alkalized cocoa powders (70 wt %) |
| 5 | Blend of extracted cocoa powders (30 wt %) and highly |
| | alkalized cocoa powders (70 wt %) |
| 6 | Extracted cocoa powders |

Observations of the sensory testing are shown in FIGS. 1A to 1F and Tables 4-5. Attributes of Samples #1 were rated by an intensity scale from 1 to 5 (i.e., 1=very low, 2=low, 3=moderate; 4=high, 5=very high). Attributes of Sample #2 through #6 were rated relative to those of Sample #1 by a relative scale from −2 to +2 (i.e., −2=much lower than sample #1, −1=lower than Sample #1, 0=about the same as Sample #1, +1=higher than Sample #1, +2=much higher than Sample #1). For example, a “−2” rating of sweetness in Sample #3 represents that the panelists regard the sweetness level in Sample #3 being much lower than the sweetness level in Sample #1. A “+2” rating of sweetness in Sample #3 represents that the panelists regard the sweetness level in Sample #3 being much higher than the sweetness level in Sample #1.

TABLE 4

| Cocoa Powder | |
|---|---|
| Sample | Remarks |
| 1 | Nice rich cocoa flavor, well balanced |
| 2 | Comparable with Sample #3 |
| 3 | Less intense compared with Sample #3 |
| 4 | There is an off note in smell, plastic like |
| 5 | Preferred |
| | Very sweet and milky, less cocoa |
| 6 | Does not disperse well; very white color |
| | Too grainy/no taste/very milky |

TABLE 5

| Cocoa Powder | No. of panelist considering |
|---|---|
| Sample | the sample to be highest in sweetness |
| 1 | 1 |
| 2 | 0 |
| 3 | 1 |
| 4 | 0 |
| 5 | 2 |
| 6 | 3 |

Further, as shown in Table 5, Sample #6 was chosen by the sensory panel as having the highest sweetness among all the sampled cocoa powders.

1. An extracted cocoa material having less bitter flavor, and a sweeter flavor, compared to an equivalent non-extracted cocoa material and has a fat content of 10-35% by weight based on a total dry weight of the extracted cocoa material.

2. The extracted cocoa material of claim 1, wherein the content of one or more bitter components in the extracted cocoa material is lower compared to an equivalent non-extracted cocoa material.

3. The extracted cocoa material of claim 2, wherein the one or more bitter components are selected from a group consisting of alkaloid, theobromine, caffeine, polyphenol, flavanol, epicatechin, catechin, epigallocatechin, proanthocianidin, and any combinations thereof.

4. The extracted cocoa material of claim 1, wherein the extracted cocoa material has a lower alkaloid content compared to an equivalent non-extracted cocoa material.

5. The extracted cocoa material of claim 1, wherein the extracted cocoa material has a lower polyphenol content compared to an equivalent non-extracted cocoa material.

6. The extracted cocoa material of claim 1, wherein the extracted cocoa material has a lower flavanol content compared to an equivalent non-extracted cocoa material.

7. The extracted cocoa material of claim 1, wherein the extracted cocoa material is an extracted cocoa powder.

8. The extracted cocoa material of claim 1, wherein the extracted cocoa material is an extracted cocoa nib, an extracted cocoa liquor, or an extracted cocoa cake and which has a fat content of 15-30 wt %, based on a total dry weight of the extracted cocoa material.

9. A food or beverage product comprising the extracted cocoa material of claim 1.

10. Use of the extracted cocoa material of claim 1 to prepare sugar-reduced or sugar-free food or beverage products.

11. A method for reducing bitter flavor and/or increasing sweet flavor in a cocoa material, comprising the steps of:
a. providing a cocoa material, preferably a cocoa nib;
b. extracting the cocoa material with a liquefied gas, at an extraction temperature from 65° C. to 110° C. and an extraction pressure to obtain an extracted cocoa material; and
c. processing the extracted cocoa material to yield an extracted cocoa powder;
wherein the extracted cocoa powder has a less bitter flavor, and a sweeter flavor, compared to an equivalent non-extracted cocoa powder.

12. The method of claim 11, wherein the liquefied gas and the cocoa material are used in a ratio of between 30:1 to 100:1.

13. The method of claim 11, wherein the extraction temperature is from 70° C. to 100° C.

14. The method of claim 11, wherein the extraction pressure is from 100 bar to 800 bar.

15. The extraction butter obtainable by the method of claim 11, which is rich in antioxidants.

16. A personal care product comprising the extraction butter of claim 15.

17. The extracted cocoa material of claim 1, wherein the extracted cocoa material has a lower theobromine and/or caffeine content compared to an equivalent non-extracted cocoa material.

18. The method of claim 11, wherein the liquefied gas is carbon dioxide.

19. The method of claim 18, wherein the carbon dioxide and the cocoa material are used in a ratio of between 30:1 to 100:1.

20. The extraction butter of claim 15, wherein the extraction butter has been subjected to heat or alkali treatment.
